(19) United States
(12) Patent Application Publication
(10) Pub. No.: US 2024/0180470 A1
(43) Pub. Date: Jun. 6, 2024

(54) Fiber Optic Enabled Stylet

(71) Applicant: Bard Access Systems, Inc., Salt Lake City, UT (US)

(72) Inventors: Steffan Sowards, Salt Lake City, UT (US); William Robert McLaughlin, Bountiful, UT (US)

(21) Appl. No.: 18/075,280

(22) Filed: Dec. 5, 2022

(51) Int. Cl. A61B 5/28

(52) U.S. Cl. CPC A61B 5/28; A61M 2025/0166

(57) ABSTRACT

A catheter placement system that includes a catheter placement device having elongate body configured for insertion into a catheter lumen, where the elongate body includes a body lumen. A compartment at a proximal end of the body is in fluid communication with the body lumen. A saline solution disposed within the compartment and the body lumen defines an electrical path along the elongate body and an electrode at a distal end of the elongate body. A stylet extends along the elongate body, where the stylet includes a multi-core optical fiber extending along the stylet, and where the multi-core optical fiber includes a number of fiber Bragg gratings disposed along the optical fiber to enable shape sensing of the optical fiber. A system module optically and electrically coupled with the catheter placement device displays a shape of the elongate body and ECG waveform.

### BACKGROUND

In the past, certain intravascular guidance of medical devices, such as guidewires and catheters for example, have used fluoroscopic methods for tracking tips of the medical devices and determining whether distal tips are appropriately localized in their target anatomical structures. However, such fluoroscopic methods expose patients and their attending clinicians to harmful X-ray radiation. Moreover, in some cases, the patients are exposed to potentially harmful contrast media needed for the fluoroscopic methods.

More recently, electromagnetic tracking systems have been used involving stylets. Generally, electromagnetic tracking systems feature three components: a field generator, a sensor unit and control unit. The field generator uses several coils to generate a position—varying magnetic field, which is used to establish a coordinate space. Attached to the stylet, such as near a distal end (tip) of the stylet for example, the sensor unit includes small coils in which current is induced via the magnetic field. Based on the electrical properties of each coil, the position and orientation of the medical device may be determined within the coordinate space. The control unit controls the field generator and captures data from the sensor unit.

Although electromagnetic tracking systems avoid line-of-sight reliance in tracking the tip of a stylet while obviating radiation exposure and potentially harmful contrast media associated with fluoroscopic methods, electromagnetic tracking systems are prone to interference. More specifically, since electromagnetic tracking systems depend on the measurement of magnetic fields produced by the field generator, these systems are subject to electromagnetic field interference, which may be caused by the presence of many different types of consumer electronics such as cellular telephones. Additionally, electromagnetic tracking systems are subject to signal drop out, depend on an external sensor, and are defined to a limited depth range. U.S. Pub. No. 2022/0034733 titled BRAGG GRATED FIBER OPTIC FLUCTUATION SENSING AND MONITORING SYSTEM, showing and describing a fiber optic shape sensing system and method, is incorporated by reference in its entirety into this application.

Disclosed herein is a catheter placement system utilizing fiber optic shape sensing and electrical signal monitoring to assist in placement of the catheter and specific placement of the tip placement of the catheter.

### SUMMARY

Disclosed herein is a catheter placement system that, according to some embodiments, includes a catheter placement device. The catheter placement device includes an elongate body configured for insertion into a catheter lumen, where the elongate body includes a body lumen extending between a body proximal end and a body distal end. The catheter placement device further includes a compartment coupled with the elongate body at the body proximal end, where the compartment is in fluid communication with the body lumen. A saline solution is disposed within the compartment and the body lumen. A stylet extends along the elongate body, and the stylet includes a multi-core optical fiber extending along the stylet, where the multi-core optical fiber includes a number of fiber Bragg gratings disposed along a length of the multi-core optical fiber.

In some embodiments, the catheter placement device is optically coupled with a system module.

In some embodiments, a distal portion of the stylet is disposed within the body lumen.

In some embodiments, the stylet includes an elongate opening extending along the distal portion, where the opening defines additional cross-sectional area of the body lumen.

In some embodiments, the system module is configured to determine a shape of the stylet based on reflected light signals emanating the fiber Bragg gratings.

In some embodiments, the system module is further configured to display an image acquired by the optical fiber.

In some embodiments, the system module is further configured to determine, based on reflected light signals emanating the fiber Bragg gratings, one or more of a temperature of the optical fiber, a motion of the optical fiber, or a displacement of fluid adjacent the optical fiber.

In some embodiments, the saline solution is electrically coupled with the system module.

In some embodiments, the stylet extends through the compartment, and a proximal portion of the stylet extends proximally away from the compartment.

In some embodiments, the proximal portion of the stylet is (i) electrically conductive, (ii) electrically coupled with the saline within the compartment, and (iii) electrically coupled with the system module.

In some embodiments, the saline solution is electrically coupled with the system module via a wire and in some embodiments, the wire extends through a sterile barrier from a sterile environment to a non-sterile environment.

In some embodiments, the catheter placement device further includes a fluid delivery device containing the saline solution, where the fluid delivery device is fluidly coupled with the compartment. In some embodiments, the compartment includes a side port, and the fluid delivery device is fluidly coupled with the compartment via the side port.

In some embodiments, the system module is configured to display an electrocardiogram waveform. In some embodiments, the system module is configured to determine a placement location of the body distal end within a patient based on the electrocardiogram waveform.

Also disclosed herein is a method that, according some embodiments, includes inserting an elongate body of a catheter placement device within a lumen of the central catheter, where the catheter placement device includes (i) a body lumen extending along the elongate body; (ii) a compartment coupled with the elongate body at a proximal end of the elongate body, where the compartment is in fluid communication with the body lumen; (iii) a saline solution disposed within the compartment and the body lumen, where the saline solution defines an electrical path along the elongate body; and (iv) a stylet extending along the elongate body, where the stylet includes a multi-core optical fiber extending along the stylet, and where the multi-core optical fiber includes a number of fiber Bragg gratings disposed along a length of the multi-core optical fiber. In such embodiments, the catheter placement device is optically and electrically coupled with a catheter placement system module. The method further includes (i) advancing the central catheter along a vasculature of the patient; (ii) determining a position of the central catheter within the patient vasculature based on a shape of the multi-core optical fiber, where the shape is determined by light reflection signals emanating from the fiber Bragg gratings; and (iv) determining a position of a distal end of the elongate body within a superior vena cava of the vasculature based on an electrocardiogram signal obtained from the patient via an electrode at the distal end.

In some embodiments of the method, the saline solution within the body lumen adjacent the distal end defines the electrode.

In some embodiments of the method, the stylet is disposed with the body lumen.

In some embodiments of the method, a syringe containing the saline solution is fluidly coupled with the compartment.

In some embodiments of the method, the saline is electrically coupled with the catheter placement system module via a wire, and the wire extends through a sterile barrier between a sterile environment and a non-sterile environment.

These and other features of the concepts provided herein will become more apparent to those of skill in the art in view of the accompanying drawings and following description, which describe particular embodiments of such concepts in greater detail.

### DRAWINGS

FIG. 1 illustrates a catheter placement system, in accordance with some embodiments.

FIG. 2 illustrates an end view of a catheter placement device of the system of FIG. 1, in accordance with some embodiments.

FIG. 3A illustrates the catheter placement system of FIG. 1 in use with a patient, in accordance with some embodiments.

FIG. 3B illustrates a detailed view of a distal portion of the catheter placement device of FIG. 1 inserted within a superior vena cava of the patient, in accordance with some embodiments.

FIG. 4 illustrates a flow chart of an exemplary method for placing a catheter within a patient, in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

The phrases “connected to,” “coupled with,” and “in communication with” refer to any form of interaction between two or more entities, including but not limited to mechanical, electrical, magnetic, electromagnetic, fluid, and thermal interaction. Two components may be coupled with each other even though they are not in direct contact with each other. For example, two components may be coupled with each other through an intermediate component.

The terms “proximal” and “distal” refer to opposite ends of a medical device, including the devices disclosed herein. As used herein, the proximal portion of a catheter placement device is the portion nearest a practitioner during use, while the distal portion is the portion at the opposite end. For example, the distal end of catheter placement device is defined as the end closest to the patient during utilization of the catheter placement device. The proximal end is the end opposite the distal end.

The term “logic” may be representative of hardware, firmware or software that is configured to perform one or more functions. As hardware, the term logic may refer to or include circuitry having data processing and/or storage functionality. Examples of such circuitry may include, but are not limited or restricted to a hardware processor (e.g., microprocessor, one or more processor cores, a digital signal processor, a programmable gate array, a microcontroller, an application specific integrated circuit “ASIC”, etc.), a semiconductor memory, or combinatorial elements.

Unless defined otherwise, all technical and scientific terms used herein have the same meaning as commonly understood by those of ordinary skill in the art. References to approximations are made throughout this specification, such as by use of the term “substantially.” For each such reference, it is to be understood that, in some embodiments, the value, feature, or characteristic may be specified without approximation. For example, where qualifiers such as “about” and “substantially” are used, these terms include within their scope the qualified words in the absence of their qualifiers. For example, where the term “substantially straight” is recited with respect to a feature, it is understood that in further embodiments, the feature can have a precisely straight configuration.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

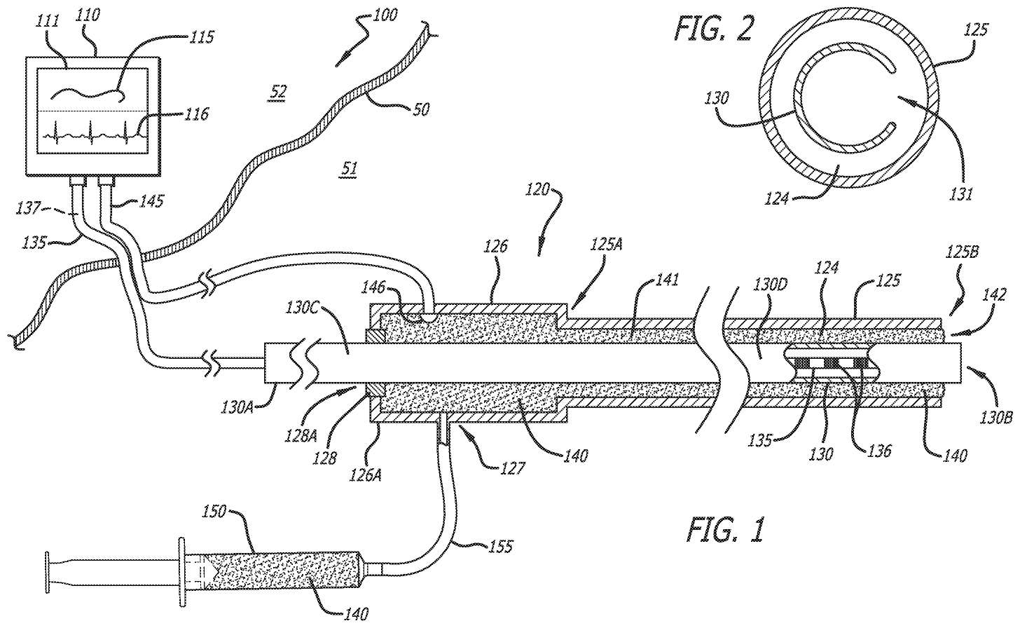

FIG. 1 illustrates a catheter placement system generally configured to assist a clinician during the placement of a catheter within a vasculature of a patient. More specifically the catheter placement system (system) 100 may (i) assist the clinician during advancement of the catheter along the vasculature and (ii) assist the clinician during placement of the distal tip of the catheter with the vasculature, such as within the superior vena cava, for example. The system 100 generally includes a catheter placement device 120 coupled with a system module 110 including a display 111.

In some instances, the system 100 may be deployed in conjunction with a sterile environment. As such, portions of the system 100 (e.g., the catheter placement device 120) may be deployed within a sterile environment 51 while other portions (e.g., the system module 110) may be deployed within a non-sterile environment 52, i.e., outside the sterile environment 51. In some instances, a sterile barrier 50 may divide the sterile environment 51 from the non-sterile environment 52. In some instances, the sterile barrier 50 may include a fabric panel, such as a drape, for example, and a number of components of the system 100 may extend through the sterile barrier 50.

The catheter placement device 120 includes an elongate body 125 that is configured for placement within a catheter lumen, such as a lumen of a central catheter, e.g., a central venous catheter (CVC) or a peripherally inserted center catheter (PICC), for example. The elongate body 125 may have sufficient length to extend between the distal tip of the catheter and an extension leg hub of the catheter. In other words, during use, a body proximal end 125A may be disposed proximal of the extension leg hub of the catheter while a body distal end 125B is disposed adjacent the distal tip of the catheter. The elongate body 125 includes a body lumen 124 extending along a length of the elongate body 125.

The catheter placement device 120 further includes a compartment 126 coupled with the elongate body at the body proximal end 125A, so that the compartment 126 is in fluid communication with the body lumen 124. A saline solution 140 is disposed within the compartment 126 and the body lumen 124. The saline solution 140 within the body lumen 124 defines an electrical path 141 extending between the body distal end 125B and the compartment 126. The body lumen 124 is open at the body distal end 125B so that the saline solution 140 may directly and electrically contact the patient. More specifically, the saline solution 140 adjacent the body distal end 125B defines a saline electrode 142, where the saline electrode 152 is configured to an acquire an electrical signal, e.g., an electrocardiogram signal, from the patient.

The catheter placement device 120 includes a fluid delivery device 150, such as a syringe, for example. The fluid delivery device 150 contains the saline solution 140. The fluid delivery device 150 is fluidly coupled with the compartment 126. In some embodiments, the fluid delivery device 150 may be coupled with the compartment 126 via a tube 155 coupled with a side port 127 of the compartment 126. During use, the fluid delivery device 150 may be provided having the saline solution 140 therein. The fluid delivery device 150 may then be coupled with the compartment 126 and the fluid delivery device 150 may dispense the saline solution 140 into the compartment 126 and along the body lumen 124.

The catheter placement device 120 further includes stylet 130 that extends along the elongate body 125. In the illustrated embodiment, the stylet 130 is disposed within the body lumen 124. However, in other embodiments, the stylet 130 may extend along an exterior of the elongate body 125 and the stylet 130 may be disposed a different lumen (not shown). The stylet 130 defines (i) a proximal portion 130C extending between a stylet proximal end 130A and the compartment 126 and (ii) a distal portion 130D extending between the compartment 126 and the stylet distal end 130B. In some embodiments, the stylet distal end 130B may be disposed adjacent the body distal end 125B. In the illustrated embodiment, the stylet 130 is generally not electrically conductive. As such, the stylet 130 may be formed a non-conductive polymeric material. In some embodiments, the distal portion 130D may include a greater stiffness than the proximal portion 130C. As the distal portion 130 is disposed along the elongate body 125 and the catheter, the greater stiffness of the distal portion 130D may assist the clinician during advancement of the catheter along the vasculature. As the proximal portion 130C is generally disposed outside the elongate body 125, the greater flexibility may allow the clinician to manipulate the catheter more easily during advancement.

The stylet 130 extends through an end wall 126A of the compartment 126. In the illustrated embodiment, the compartment 126 includes a coupling member 128 configured to sealably couple the stylet 130 to the compartment 126. The coupling member 128 includes an opening 128A through which the stylet 130 is disposed. In some embodiments, the coupling member 128 may couple the stylet 130 to the compartment 126 such that longitudinal displacement of the stylet 130 with respect to the compartment 126, including the elongate body 125, is prevented. In other embodiments, the coupling member 128 may couple the stylet 130 to the compartment 126 such that slidable displacement of the stylet 130 with respect to the compartment 126 is allowed. In some embodiments, the coupling member 128 may include an elastomeric septum and the opening 128A may be a slit extending through the septum.

In the illustrated embodiment, the catheter placement device 120 includes a wire 145 extending between the compartment 126 and the system module 110. A wire electrode 146 electrically couples the wire 145 to the saline solution 140 within the compartment 126. By way of summary, during use, the anatomy of the patient adjacent the body distal end 125B is electrically coupled with the system module 110 via the saline electrode 142, the saline solution 140 along the body lumen 124, the saline solution 140 within the compartment 126, the wire electrode 146, and the wire 145. In some instances, the wire 145 may extend through the sterile barrier 50.

The stylet includes an optical fiber 135 extending along the stylet 130. The optical fiber 135 is a multi-core optical fiber including a number of fiber Bragg gratings 136 disposed along a length of the optical fiber 135. The optical fiber 135 is configured to enable shape sensing of the optical fiber 135 based on reflected light signals emanating (i.e., reflected by) the fiber Bragg gratings 136. As the optical fiber 135 extends along the stylet 130, as the stylet 130 extends along the elongate body 125, and as the elongate body 125 is disposed within a lumen of the catheter, and the optical fiber 135 enables shape sensing of the catheter.

In some embodiments, the fiber Bragg gratings 136 may be configured to define light reflection signals based on conditions of the optical fiber 135, such as a temperature of the optical fiber 135, a motion of the optical fiber 135, or a displacement of fluid adjacent the optical fiber 135, such as via the Doppler effect, for example. Although not shown, in some embodiments, the optical fiber 135 may extend distally beyond the stylet distal end 130B.

In some embodiments, the optical fiber 135 may be configured to project illuminating light away from the stylet distal end 130B and receive imaging light entering the optical fiber 135 at the stylet distal end 130B. As such, the optical fiber 135 may obtain images of anatomical elements of the patient adjacent the stylet distal tip 130B during use.

In the illustrated embodiment, the optical fiber 135 extends away from the proximal portion 130C of the stylet 130 so that the optical fiber 135 may optically couple with the system module 110, such as via an optical connection member (not shown), for example. In some instances, the optical fiber 135 or optical connection member may extend through the sterile barrier 50.

The system module 110 is operatively coupled with the catheter placement device 120. More specifically, the system module 110 is (i) optically coupled with the optical fiber 135 and (ii) electrically coupled with the saline solution 140. In the illustrated embodiment, the system module 110 is configured to provide information pertaining to the placement of the catheter to the clinician such as displaying information and/or images (i.e., depicting images or information on the display 111). The system module 110 may include a console having a number of processors and logic stored in memory (e.g., a non-transitory computer-readable medium). The logic, when executed by the processors, governs the operation of the system module 110. The system module 110 further include a light source and an optical receiver.

The system module 110 is configured to (i) determine, based on reflected light signals emanating the fiber Bragg gratings 136, a shape 115 of the optical fiber 135, where the shape 115 represents the shape of the catheter, and (ii) display the shape 115, i.e., render an image of the shape 115 on the display 111. In some embodiments, the system module 110 may display the shape 115 in real time during advancement of the catheter along the vasculature to assist the clinician in placement of the catheter.

The system module 110 is configured to receive electrical signals from the catheter placement device 120, where in some instances, the electrical signals emanate from the patient, such as an electrocardiogram (ECG) signal, for example. In some embodiments, the system module 110 may a display an ECG waveform 116. In some instances, the ECG waveform 116 may vary in accordance with the location of the body distal tip 125B within the vasculature, such as within the superior vena cava, for example. In some embodiments, the logic may compare the ECG waveform 116 with an ECG waveform stored in memory, where the ECG waveform stored in memory is consistent with placement of the body distal end 125B within the lower one-third of the superior vena cava. As a result of the comparison, the logic may determine that the body distal end 125B is located within the lower one-third of the superior vena cava. Generally stated, the system module 110 may be configured to determine a placement location of the body distal end 125B (and, by association, the distal tip of the catheter) within a patient vasculature based on the electrocardiogram signal.

According to another embodiment, the proximal portion 130C of the stylet 130 may be electrically conductive defining an electrical path in leu of the wire 145 and the wire electrode 146. In such an embodiment, the optical fiber 135 and/or the optical connecting member may include an electrical conducting element 137 extending therealong. The electrical conducting element 137 may be electrically coupled with the proximal portion 130C and extend between the proximal portion 130C and system module 110. By way of summary, during use, the anatomy of the patient adjacent the body distal end 125B is electrically coupled with the system module 110 via the saline electrode 142, the saline solution 140 along the body lumen 124, the saline solution 140 within the compartment 126, the proximal portion 130C, and the electrical conducting element 137.

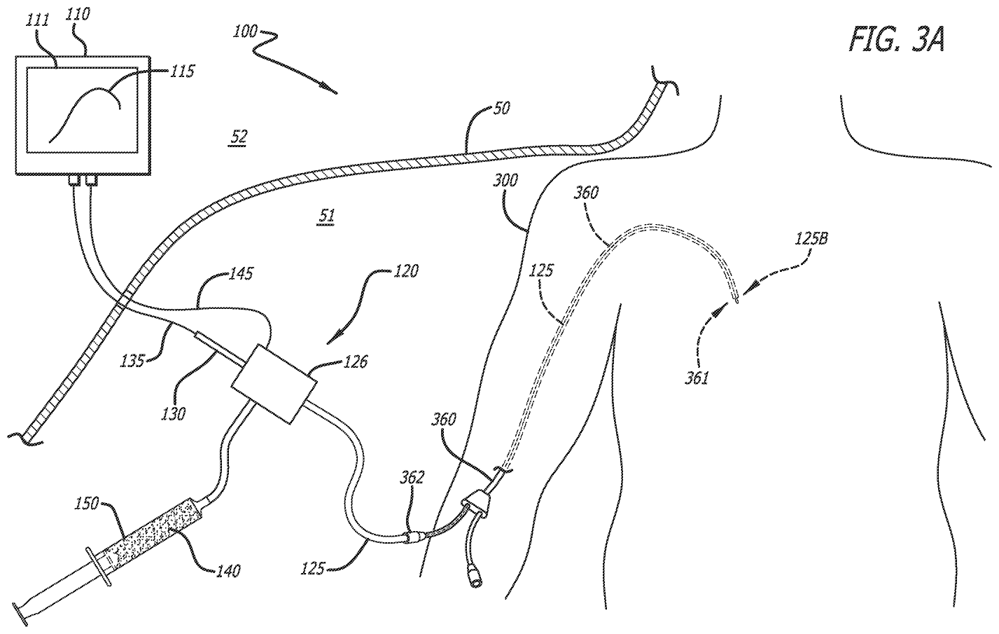

FIG. 2 is a distal end view of the elongate body 125 including an end view of the distal portion 130D of the stylet 130. To maximize cross-section area of the body lumen 124, the distal portion 130D may include an elongate opening 131 (i.e., a grove or slot) extending along the distal portion 130D. As such, the cross-sectional area of the elongate opening 131 adds to the annular cross-sectional area of the lumen 124.

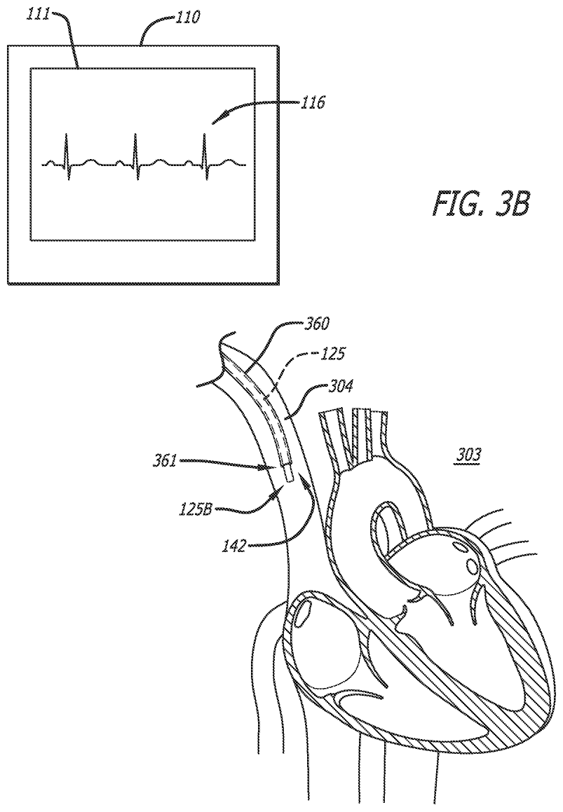

FIG. 3A illustrates the system 100 in use with a patient 300. A catheter 360 (e.g., a PICC in the illustrated instance) is advanced along a vasculature of the patient 300. The elongate body 125 of the catheter placement device 120 is inserted into a lumen of the catheter 360 via a hub 362 of an extension leg of the catheter 360. The body distal end 125B is disposed adjacent a distal tip 361 of the catheter 360. The fluid delivery device 150 contains the saline solution 140 and the saline solution 140 is dispensed into the compartment 126 and along the lumen 124 (FIG. 1). The sterile barrier 50 is disposed between the system module 110 and the catheter placement device 120, where the optical fiber 135 and the wire 145 pass through the sterile barrier 50. The system module 110 renders an image of the shape 115 on the display 111.

Referring now to FIG. 3B, a detailed view of distal portion of the elongate body 125, where the body distal end 125B and the distal tip 361 of the catheter 360 are disposed within the superior vena cava 304, is shown in accordance with some embodiments. The system module 110 including the display 111 is also shown. FIG. 3B illustrates a detailed perspective of the vasculature proximate to the heart 303 of a patient 300 as well as the anatomy of the heart 303. The saline electrode 142 is located with the superior vena cava 304 so as to obtain an ECG signal from the heart 303. The waveform 116 of the ECG signal is depicted on the display 111.

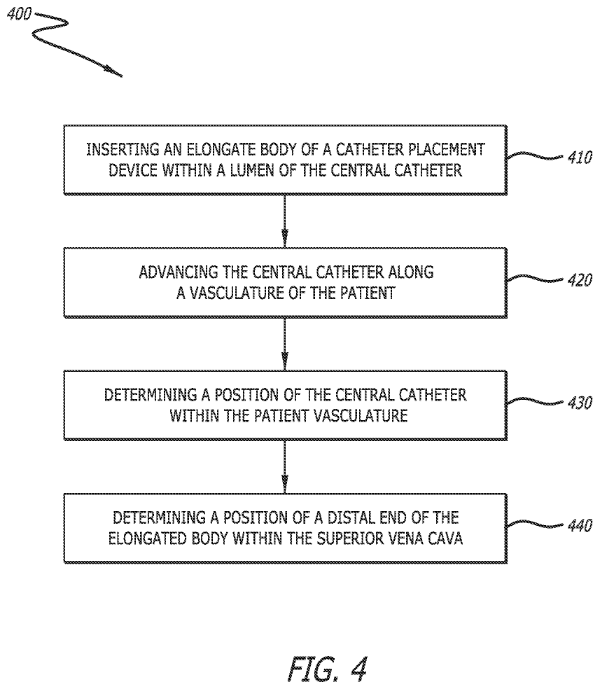

FIG. 4 illustrates a flow chart of an exemplary method for placing a central catheter within a patient that may include all or any subset of the following steps, actions, or processes. The method 400 may include inserting an elongate body of the catheter placement device within the lumen of the central catheter (block 410). The catheter placement device is optically and electrically coupled with a catheter placement system module.

In accordance with the method 400, the catheter placement device includes the body lumen extending along the elongate body and the compartment coupled with the elongate body at a proximal end of the elongate body, where the compartment is in fluid communication with the body lumen. The saline solution is disposed within the compartment and the body lumen, such that the saline solution defines the electrical path along the elongate body. In some embodiments of the method 400, a syringe containing the saline solution is fluidly coupled with the compartment. In some embodiments of the method 400, the saline solution within the body lumen adjacent the distal end defines the electrode. In some embodiments of the method 400, the saline solution is electrically coupled with the system module via the wire, and the wire extends through a sterile barrier between a sterile environment and a non-sterile environment.

The stylet extends along the elongate body, where the stylet includes a multi-core optical fiber extending along the stylet, and where the multi-core optical fiber includes a number of fiber Bragg gratings disposed along a length of the multi-core optical fiber. In some embodiments of the method 400, the stylet is disposed with the body lumen.

The method 400 further includes advancing the central catheter along the vasculature of the patient (block 420). The method 400 may further include determining the position of the central catheter within the patient vasculature (block 430) based on a shape of the multi-core optical fiber, where the shape is determined by light reflection signals emanating from the fiber Bragg gratings. The method 400 may further include determining a position of the distal end of the elongate body within the superior vena cava (block 440) based on an electrocardiogram signal obtained from the patient via the saline electrode at the distal end.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

What is claimed is:

1. A catheter placement system, comprising:
a catheter placement device, comprising:
an elongate body configured for insertion into a catheter lumen, the elongate body including a body lumen extending between a body proximal end and a body distal end;
a compartment coupled with the elongate body at the body proximal end, the compartment in fluid communication with the body lumen;
a saline solution disposed within the compartment and the body lumen; and
a stylet extending along the elongate body, stylet including a multi-core optical fiber extending along the stylet, the multi-core optical fiber including a number of fiber Bragg gratings disposed along a length of the multi-core optical fiber.

2. The system according to claim 1, wherein a distal portion of the stylet is disposed within the body lumen.

3. The system according to claim 2, wherein the stylet includes an elongate opening extending along the distal portion, the opening defining additional cross-sectional area of the body lumen.

4. The system according to claim 1, wherein the catheter placement device is optically coupled with a system module.

5. The system according to claim 4, wherein the system module is configured to determine a shape of the stylet based on reflected light signals emanating the fiber Bragg gratings.

6. The system according to claim 5, wherein the system module is configured to display the shape.

7. The system according to claim 4, wherein the system module is further configured to display an image acquired by the optical fiber.

8. The system according to claim 4, wherein the system module is further configured to determine, based on the reflected light signals emanating the fiber Bragg gratings, one or more of:
a temperature of the optical fiber,
a motion of the optical fiber, or
a displacement of fluid adjacent the optical fiber.

9. The system according to claim 4, wherein the saline solution is electrically coupled with the system module and the patient.

10. The system according to claim 9, wherein the saline solution is electrically coupled with the system module via a wire.

11. The system according to claim 10, wherein the wire extends through a sterile barrier between a sterile environment and a non-sterile environment.

12. The system according to claim 9, wherein the system module is configured to determine an electrocardiogram signal of the patient and display an electrocardiogram waveform.

13. The system according to claim 12, wherein the system module is configured to determine a placement location of the body distal end within a vasculature of the patient based on the electrocardiogram signal.

14. The system according to claim 1, wherein:
the stylet extends through the compartment, and
a proximal portion of the stylet extends proximally away from the fluid compartment.

15. The system according to claim 14, wherein the proximal portion of the stylet is:
electrically conductive,
electrically coupled with the saline solution within compartment, and
electrically coupled with the system module.

16. The system according to claim 1, wherein the catheter placement device further comprises a fluid delivery device containing the saline solution, the fluid delivery device fluidly coupled with the compartment.

17. The system according to claim 16, wherein:
the compartment includes a side port, and
the fluid delivery device is fluidly coupled with the compartment via the side port.

18. A method of place a central catheter within a patient, comprising:
inserting an elongate body of a catheter placement device within a lumen of the central catheter, the catheter placement device comprising:
a body lumen extending along the elongate body;
a compartment coupled with the elongate body at a proximal end of the elongate body, the compartment in fluid communication with the body lumen;
a saline solution disposed within the compartment and the body lumen, the saline solution defining an electrical path along the elongate body; and
a stylet extending along the elongate body, the stylet including a multi-core optical fiber extending along the stylet, the multi-core optical fiber including a number of fiber Bragg gratings disposed along a length of the multi-core optical fiber,
wherein the catheter placement device is optically and electrically coupled with a catheter placement system module;
advancing the central catheter along a vasculature of the patient;
determining a position of the central catheter within the vasculature based on a shape of the multi-core optical fiber, the shape determined by light reflection signals emanating from the fiber Bragg gratings; and
determining a position of a distal end of the elongate body within a superior vena cava of the vasculature based on an electrocardiogram signal obtained from the patient via an electrode at the distal end.

19. The method according to claim 18, wherein the saline solution within the body lumen adjacent the distal end defines the electrode.

20. The method according to claim 18, wherein the stylet is disposed within the body lumen.

21. The method according to claim 18, wherein the catheter placement device includes a syringe containing the saline solution, the syringe fluidly coupled with the compartment.

22. The method according to claim 18, wherein:
the saline is electrically coupled with the catheter placement system module via a wire, and
the wire extends through a sterile barrier between a sterile environment and a non-sterile environment.
